(19) United States
(12) Patent Application Publication
(10) Pub. No.: US 2021/0298413 A1
(43) Pub. Date: Sep. 30, 2021

(54) SMART LIFTING SHOES FOR TAKING EXERCISE

(71) Applicant: Kang Su YOU, Busan (KR)

(72) Inventor: Kang Su YOU, Busan (KR)

(21) Appl. No.: 17/087,892

(22) Filed: Nov. 3, 2020

(30) Foreign Application Priority Data
Mar. 31, 2020 (KR) 1020200039223

(51) Int. Cl. A43B 13/14; G01L 5/161

(52) U.S. Cl. CPC A43B 13/14; G01L 5/161

(57) ABSTRACT

Proposed is smart lifting shoes for taking exercise, each shoe including: an outsole configured to have combination through-holes that correspond to portions, respectively, of the sole, to have an accommodation groove in a center of an upper part thereof, the center corresponding to a center of the sole, and to have a connection groove; sensor units within upper portions of the combination through-holes, respectively; a controller including: a pressure reception module within the accommodation groove and receiving pressure information measured by the sensor units; a balance measurement module computing a balance of the sole on the basis of the pressure information and generating balance information; a power supply module supplying power; and a communication module transmitting the pressure information and the balance information; and adjustment enabling portions, each having a terminal portion on an upper part thereof, that are closely connected into the combination through-holes, respectively.

### CROSS REFERENCE TO RELATED APPLICATION

The present application claims priority to Korean Patent Application No. 10-2020-0039223, filed on March 31, the entire contents of which is incorporated herein for all purposes by this reference.

### BACKGROUND

### Field

The present disclosure relates to smart lifting shoes for taking exercise. More particularly, the present disclosure relates to smart lifting shoes for taking exercise, each of which includes sensors, a controller, and adjustment enabling portions in order for a user to maintain his/her balance. The sensors are installed within four portions of an outsole of the smart lifting shoes. The controller receives pressure information measured by the sensors and performs computation to generate balance information. In order for the user to correct his/her improper balance on the basis of the balance information of the user, the adjustment enabling portions are closely connected into portions, respectively, of the outsole, whereby the weight of the user is concentrated on the portions of the outsole.

### Description of Related Art

Generally, when a user stands in a proper posture for maintaining his/her balance for the center of mass in motion or the center of gravity, the weight of the user is concentrated on his/her soles. That is, the weight of the user is concentrated on distal phalanx (first phalange) portions, first metatarsal bone head portions, fifth metatarsal bone head portions, and calcaneus portions of his/her soles.

Generally, there are four states to consider in gait analysis. In a stand state, a right leg of the user is properly supported on the ground. In a toe-off state, the leg of the user is moved backward with respect to an upper body of the user and at the same time the leg lifts off the ground. In a middle swing state, the leg of the user lifts up from the ground and then is moved from backward to forward. In a heel strike state, the leg of the user starts to land on the ground after being moved forward with respect to the upper body. The standing state in gait analysis is important for a weightlifter who puts on shoes. In order for the user to maintain his/her balance, the weight of the user is properly distributed over the distal phalanx (first phalange) portions, the first metatarsal bone head portions, the fifth metatarsal bone head portions, and the calcaneus portions of his/her soles, on which the weight of the user is concentrated. To this end, the thicknesses of insoles of shoes in the related art are changed to adjust heights of the shoes. Alternatively, adjustment enabling portions, each with a predetermined thickness, are installed in insoles and outsoles of shoes in the related art. However, the user cannot maintain his/her balance according to his/her type of body. The shoes in the related art have a function of measuring pressure against the left feet and right feet of the user but do not have a function of measuring a balance between the left feet and right feet supported on the ground. This balance function is important in a standing posture. A disadvantage of shoes in the related art is that such shoes are not suitable in the standing posture.

Therefore, it is necessary to develop lifting shoes that are capable of determining to which portion of an outsole of the shoe pressure is applied in the standing posture and of providing guidance and information for adjusting a height of the outsole according to a change in the pressure against the outsole of the shoe and against the ground. To this end, sensors measuring the pressure need to be installed in the outsole of the shoe.

The foregoing is intended merely to aid in the understanding of the background of the present disclosure, and is not intended to mean that the present disclosure falls within the purview of the related art that is already known to those skilled in the art.

### Documents of Related Art

Korean Patent No. 10-1856077 (GAIT CORRECTION SYSTEM UTILIZING SMART INSOLE)

Korean Patent No. 10-1730311 (SHOCK OBSORPTION TUBE SYSTEM FOR SHOES)

### SUMMARY

An objective of the present disclosure is to provide smart lifting shoes for taking exercise, which are capable of maintaining user's balance in a standing posture. In the smart lifting shoes, a pressure applied to a preset portion of a sole is measured with respect to the center of gravity of the user. Then, a portion of the sole in which a change in pressure is required for the user to maintain his/her balance is determined according to a result of the measurement. Then, an adjustment enabling portion for adjusting a height from the ground is combined with a portion of an outsole that corresponds to the portion of the sole.

According to an aspect of the present disclosure, there is provided smart lifting shoes for taking exercise, each of which is configured from an outsole to be brought into contact with the ground, a midsole combined with an upper portion of the outsole, an insole combined with an upper portion of the midsole to be brought into contact with a sole of a user, and an upper combined with the outsole to accommodate a foot of the user, the smart lifting shoes each including an outsole configured to have first, second, third, and fourth combination through-holes in positions thereon that correspond to a distal phalanx portion, a first metatarsal bone portion, a fifth metatarsal bone portion, and a calcaneus portion, respectively, of the sole, to have an accommodation groove in a center of an upper part thereof, the center thereof corresponding to a center of the sole, and to have a connection groove that connects between the accommodation groove and the fourth combination through-hole; sensor units installed within upper portions of the first, second, third, and fourth combination through-holes, respectively; a controller including: a pressure reception module mounted within the accommodation groove for operation in conjunction with the sensor units and receiving pressure information measured by the sensor units; a balance measurement module computing a balance of the sole on the basis of the pressure information and generating balance information; a power supply module supplying power; and a communication module transmitting the pressure information and the balance information; and adjustment enabling portions, each having a terminal portion on an upper part thereof, that are closely connected into the first, second, third, and fourth combination through-holes, respectively, in the outsole, the terminal portion serving for contact with each of the sensor units.

In the smart lifting shoes, the adjustment enabling portions having different lengths that are determined on the basis of the balance information provided by the controller may be closely connected into the first, second, third, and fourth combination through-holes in the outsole.

In the smart lifting shoes, the terminal portion combined with a center of an upper part of one of the adjustment enabling portions may have a contact tab protruding from a center of an upper portion thereof, and a protrusion jaw protruding from a lower cylindrical end thereof, and each of the adjustment enabling portions may have a cylindrical insertion groove in the upper part and may have a cylindrical edge portion, a cylindrical space groove, and a reinforcement tab in an external circumferential end portion, the protrusion jaw of the terminal portion 500 being inserted into the cylindrical insertion groove for combination, the cylindrical space groove being inwardly positioned next to the cylindrical edge portion the reinforcement tab being positioned between the cylindrical space groove and the cylindrical insertion groove.

In the smart lifting shoes, a plurality of air flow through-holes may pass through each of the adjustment enabling portion downward from a bottom of the cylindrical space groove in such a manner as to be positioned a distance away from each other, each of the adjustment enabling portions may have a net portion on a lower surface, the net portion having a plurality of grid grooves arranged in matrix form and communicating with the air flow through-holes, a cylindrical end portion of each of the adjustment enabling portions, which is positioned outward from the air flow through-holes in a lower surface of the adjustment enabling portion, may be bonded to an external circumferential end portion of the net portion for connection, and an internal surface of each of the adjustment enabling portions, which is positioned inward from the air flow through-holes, and a corresponding internal surface of an upper portion of the net portion may be brought into contact with each other without being bonded with each other.

In the smart lifting shoes, each of the adjustment enabling portions may have an arc-shaped first insertion hole extending horizontally into an external circumferential surface of a lower part, the first insertion hole being open at a first surface and being closed at a second surface facing the first surface, each of the adjustment enabling portions may have a vertical second insertion hole in a center of an upper part, the vertical second insertion hole communicating with a center of the first insertion hole from top down, a vertical support bar may be connected to a center of a lower surface of the terminal portion and may be inserted into the second insertion hole in the adjustment enabling portion, a sphere-shaped rotation guidance ball may be connected to a lower end of the vertical support bar, and a load support plate inserted into the first insertion hole in each of the adjustment enabling portions may have a semi-sphere-shaped support groove in a center thereof, the rotation guidance ball of the terminal portion 500 being accommodated within the semi-sphere-shaped support groove.

According to the present disclosure, a pressure applied to a preset portion of a sole is measured in real time with respect to the center of gravity of a user in a standing posture. Then, a portion of the sole in which a change in pressure is required for the user to maintain his/her balance is determined according to a result of the measurement. Then, an adjustment enabling portion for adjusting a height is combined with a portion of an outsole that corresponds to the portion of the sole. In a state where the user puts on the smart lifting shoes for taking exercise, the user can accordingly maintain his/her balance in the standing posture. Furthermore, unlike in the case of a sole of a shoe in the related art, the measurement and the correction can be made whenever needed. Accordingly, imbalance of a pressure against a foot of the user that occurs due to wear and tear of the shoe, a change of a shape of the shoe, and the like can be annulled.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a conceptual view illustrating portions of a sole, on which the weight of a user is concentrated;

FIG. 2 is a view illustrating sensor units and a controller that are positioned in an outsole positioned under an insole in smart lifting shoes for taking exercise according to an embodiment of the present disclosure;

FIG. 3 is a view illustrating that the controller is accommodated within an accommodation groove and sensor units and adjustment enabling portions are connected into combination through-holes in the smart lifting shoes for taking exercise according to the embodiment of the present disclosure;

FIG. 4 is a view illustrating a state of a bottom surface of the outsole in the smart lifting shoes for taking exercise according to the embodiment of the present disclosure;

FIG. 5 is a conceptual block diagram illustrating a functional relationship among the sensor unit, the controller, and a display unit in the smart lifting shoes for taking exercise according to the embodiment of the present disclosure;

FIGS. 6A is a view illustrating a state where the adjustment enabling portion and the terminal portion are separated from each other in the smart lifting shoes for taking exercise according to the embodiment of the present disclosure;

FIGS. 6B is a view illustrating a state where the adjustment enabling portion and the terminal portion are assembled in the smart lifting shoes for taking exercise according to the embodiment of the present disclosure;

FIG. 7A and 7B are respectively a perspective view and a cross-sectional view illustrating the adjustment enabling portion having air flow through-holes and a net portion in the smart lifting shoes for taking exercise according to the embodiment of the present disclosure;

FIG. 8A is a view illustrating that the terminal portion having a support bar and a rotation guidance ball, and a load support plate are removed from first and second insertion holes, respectively, in the adjustment enabling portion in the smart lifting shoes for taking exercise according to the embodiment of the present disclosure;

FIG. 8B is a cross-sectional view illustrating that the terminal portion having the support bar and the rotation guidance ball, and the load support plate are inserted into the first and second insertion holes, respectively, in the adjustment enabling portion in the smart lifting shoes for taking exercise according to the embodiment of the present disclosure;

FIG. 9 is a cross-sectional view illustrating that a stability maintenance portion is combined with a protruding part of the adjustment enabling portion in the smart lifting shoes for taking exercise according to the embodiment of the present disclosure.

### DETAILED DESCRIPTION OF THE INVENTION

An embodiment of the present disclosure will be described in detail below with reference to the accompanying drawings.

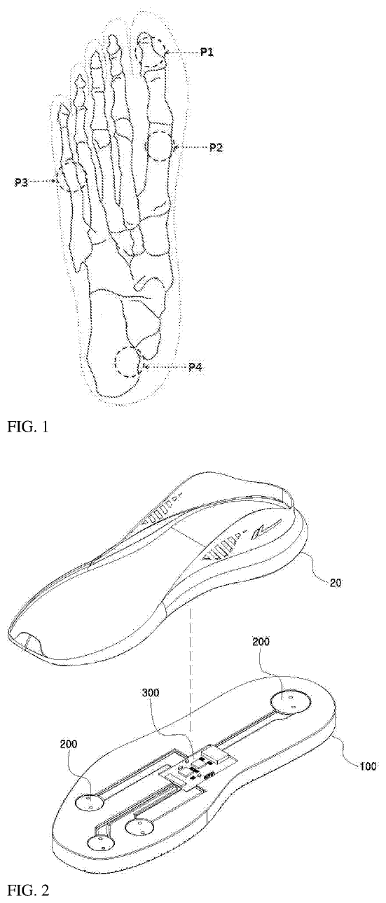

Lifting shoes (weightlifting shoes) according to the present disclosure, although not illustrated, are shoes that are put on by a user who do an action or exercise that needs to maintain a balance for the weight of the user or a balance for the weight of the user and the weight of athletic equipment. Examples of the lifting shoes are lifting shoes that are put on by a weightlifter, but the present disclosure is not limited to the lifting shoes. When the user who puts on customized lifting shoes for the weightlifter rises to his/her feet, that is, takes a standing posture that is one of postures for gait analysis, the weight of the user is supported in an evenly distributed manner on his/her left foot and right foot, and thus his/her body balance is maintained. At this time, as illustrated in FIG. 1, most of the weight is concentrated on a distal phalanx portion P1, a first metatarsal bone portion P2, a fifth metatarsal bone portion P3, and a calcaneus portion P4 that are portions of a sole of each of the left foot and right foot. The first metatarsal bone portion P2 is adjacent to a big toe, and the fifth metatarsal bone portion P3 is adjacent to a little toe.

That is, the first and fifth metatarsal bone portions P2 and P3 correspond to convexly protruding portions of the sole, which are positioned below first and fifth metatarsal bones, respectively.

Accordingly, a height of an outsole 100 that constitutes each of the smart lifting shoes according to the present disclosure, from the ground is adjusted using individually the selected portions P1, P2, P3, and P4. The smart lifting shoes stabilize a change in the center of gravity in a more balanced manner when the weightlifter lifts a barbell in the standing posture.

Smart lifting shoes for taking exercise according to the present disclosure will be described in detail below.

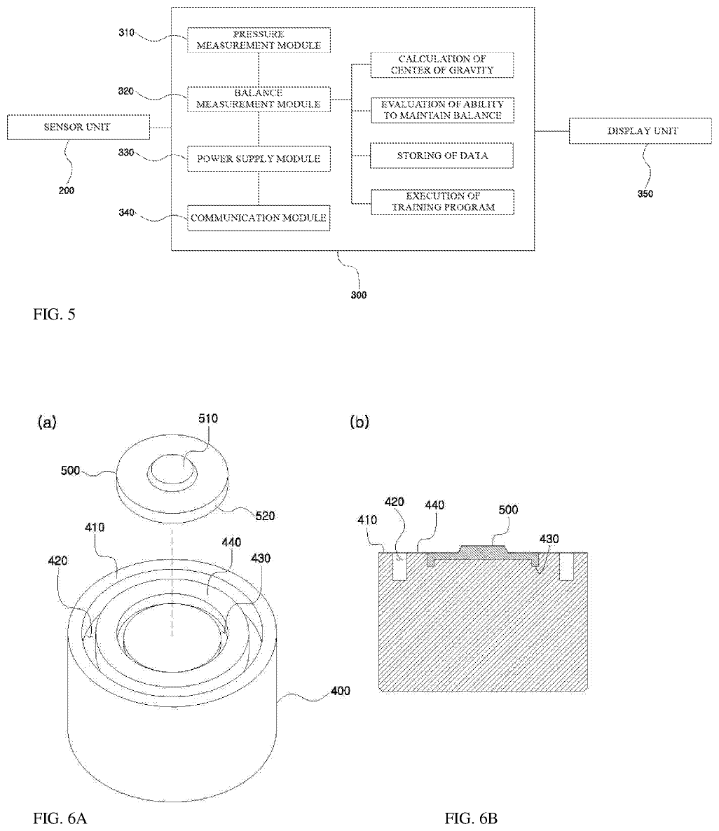

As illustrated in FIGS. 1 to 5, smart lifting shoes for taking exercise (hereinafter referred to “lifting shoes”) according to an embodiment of the present disclosure are each configured from an outsole to be brought into contact with the ground, a midsole combined with an upper portion of the outsole, an insole combined with an upper portion of the midsole to be brought into contact with a sole, and an upper combined with the outsole to accommodate a foot. The smart lifting shoe includes an outsole 100, sensor units 200, a controller 300, and adjustment enabling portions 400. The outsole 100 has first, second, third, and fourth combination through-holes 110a, 110b, 110c, and 110d, in positions thereon that correspond to a distal phalanx portion P1, a first metatarsal bone portion P2, a fifth metatarsal bone portion P3, and a calcaneus portion P4, respectively, of the sole. The outsole 100 has an accommodation groove 101 in the center of an upper part, the center thereof corresponding to the center of the sole. The outsole 100 has a connection groove 102 that connects between the accommodation groove and the fourth combination through-hole 110d. The sensor units are installed within upper portions of the first, second, third, and fourth combination through-holes 110a, 110b, 110c, and 110d, respectively. The controller 300 includes: a pressure reception module 310 mounted within the accommodation groove 101 for operation in conjunction with the sensor units 200 and receiving pressure information measured by the sensor units 200; a balance measurement module 320 computing a balance of the sole on the basis of the pressure information and generating balance information; a power supply module 330 supplying power; and a communication module 340 transmitting the pressure information and the balance information. The adjustment enabling portions 400, each having a terminal portion 500 on an upper part, that are closely connected into the first, second, third, and fourth combination through-holes 110a, 110b, 110c, and 110d, respectively, in the outsole 100, the terminal portion 500 serving for contact with each of the sensor units 200.

Although not illustrated, each of the lifting shoes is configured from the outsole 100, the midsole 20, the insole, and the upper. The outsole 100 includes the sensor units 200, the controller 300, and the adjustment enabling portions 400.

As illustrated in FIGS. 1 and 2, for description, the outsole 100 is divided into an upper portion facing the midsole 20 and a lower portion facing the ground. The outsole 100 has first, second, third, and fourth combination through-holes 110a, 110b, 110c, and 110d from the top down on the upper part. The first, second, third, and fourth combination through-holes 110a, 110b, 110c, and 110d are positioned in positions thereon that correspond to the distal phalanx portion P1, the first metatarsal bone portion P2, the fifth metatarsal bone portion P3, and the calcaneus portion P4, respectively, of the sole.

That is, a position of the first combination through-hole 110a in the outsole 100 corresponds to that of the distal phalanx P1. A position of the second combination through-hole 110b in the outsole 100 corresponds to that of the first metatarsal bone portion P2. A position of the third combination through-hole 110c in the outsole 100 corresponds to that of the fifth metatarsal bone portion P3. A position of the fourth combination through-hole 110d in the outsole 100 corresponds to that of the calcaneus portion P4.

The center of the upper portion of the outsole 100 corresponds to the center of the sole. Through the accommodation groove 101 in the center of the upper portion of the outsole 100 and the connection groove 102, the first, second, third, and fourth combination through-holes 110a, 110b, 110c, and 110d communicate with each other. The connection groove 102 serves to guide a connection line 201 that connects between the controller 300 accommodated within the accommodation groove 101 and each of the sensor units 200 installed in the first, second, third, and fourth combination through-holes 110a, 110b, 110c, and 110d, respectively. The connection line 201 is a wire along which data measured by the sensor units 200 is transmitted or power is supplied to the sensor units 200. The outsole 100 may be manufactured without the connection groove 102. In this case, wireless communication is performed between each of the sensor units 200 and the controller 300, and the sensor units 200 have their respective power supply sources.

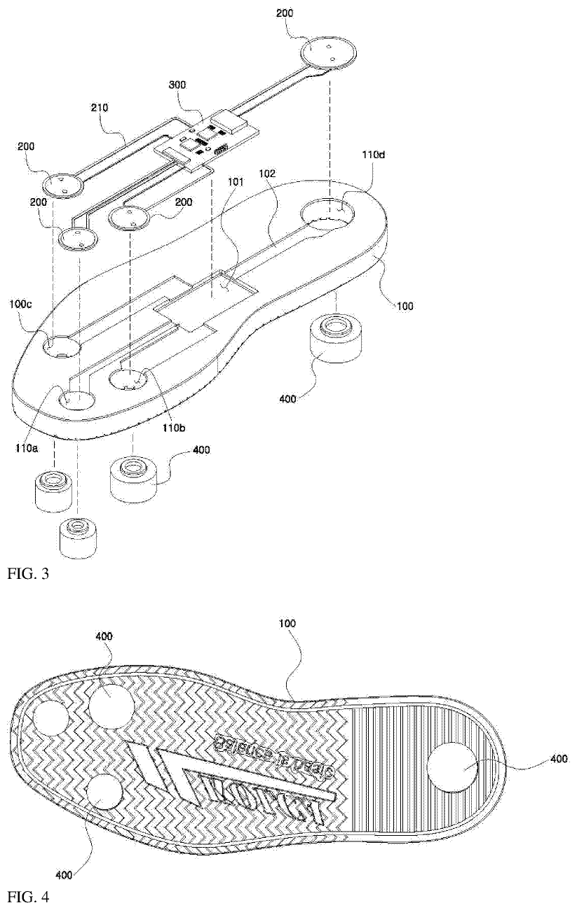

As illustrated in FIGS. 2 and 3, the sensor units 200 are installed within the upper portions of the first, second, third, and fourth combination through-holes 110a, 110b, 110c, and 110d in the outsole 100. The sensor units 200 are force sensing resistors (FSR), load cells, or the like that measure a pressure, a weight, and a position. The sensor units 200 that are to be installed within the upper portions of the first, second, third, and fourth combination through-holes 110a, 110b, 110c, and 110d, respectively, have sizes that correspond to areas of the upper portions of the first, second, third, and fourth combination through-holes 110a, 110b, 110c, and 110, respectively, for close connection.

As illustrated in FIGS. 2 to 4, the terminal portions 500 are provided on the upper parts, respectively, of the adjustment enabling portions 400 for connection to the sensor units 200 and are closely connected into the first, second, third, and fourth combination through-holes 110a, 110b, 110c, and 110d, respectively, in the outsole 100. The terminal portions 500 are brought into contact with the sensor units 200, respectively. Thus, the sensor units 200 measure weights applied to the adjustment enabling portions 400. The sensor units 200 measure the weight that is supported on the soles of user's feet, through the adjustment enabling portions 400 closely connected into the first, second, third, and fourth combination through-holes 110a, 110b, 110c, and 110d corresponding to the portions Pl, P2, P3, and P4, respectively. The adjustment enabling portions 400 will be described below. Before the user's feet are fitted with the lifting shoes put on by the user, lower ends of the adjustment enabling portions 400 inserted into the first, second, third, and fourth combination through-holes 110a, 110b, 110c, and 110d, respectively, have the same height as a bottom surface that is a lower surface of the outsole 100. Thereafter, in a case where correction is necessary, on the basis of weight information provided by the sensor units 200, it is determined which of the first, second, third, and fourth combination through-holes 110a, 110b, 110c, and 110d is associated with an abnormality on a weight distribution graph or a user's balance distribution graph. A lower part of the adjustment enabling portion closely connected into the combination through-hole associated with the abnormality protrudes from the lower surface of the outsole 100.

As illustrated in FIG. 4, the controller 300 includes the pressure reception module 310, the balance measurement module 320, the power supply module 330, and the communication module 340. The pressure reception module 310, mounted within the accommodation groove 101 for operation in conjunction with the sensor units 200, receives the weight information measured by the sensor units 200. The balance measurement module 320 computes the balance of the sole on the basis of the weight information and generates balance information. The power supply module 330 supplies power. The communication module 340 transmits the weight information and the balance information.

First, the adjustment enabling portions 400 are closely connected into the first, second, third, and fourth combination through-holes 110a, 110b, 110c, and 110d, respectively, in such a manner that the lower ends of the adjustment enabling portions 400 closely connected into the first, second, third, and fourth combination through-holes 110a, 110b, 110c, and 110d, respectively, have the same height as the bottom surface that is the lower surface of the outsole 100.

When the user who puts on the lifting shoes takes the standing posture, the sensor units 200 measure pressure applied to the adjustment enabling portions 400 closely connected into the first, second, third, and fourth combination through-holes 110a, 110b, 110c, and 110d, respectively. The sensor units 200 transmit pressure information to the pressure reception module 310 of the controller 300 that operates in conjunction with the sensor units 200. The balance measurement module 320 of the controller 300 measures a pressure distribution over the user's sole on the basis of the transmitted pressure information and analyzes the pressure.

The controller 300 may further include an output module 360 that operates in conjunction with the controller 300 under the control of the controller 300. The output module 360 provides information for feedback in the form of vibration or audio according to information on the pressure distribution over the user's sole, which results from analyzing the pressure.

Specifically, when a predetermined condition (a condition for determining whether or not the pressure distribution over the portions of the sole deviates from a balance posture) is satisfied, feedback that the user's posture is required to be corrected is provided in real time under the control of the controller 300.

The controller 300 may include a wireless communication module 340 that wirelessly receives the pressure information measured by the sensor units 200.

The controller 300 analyzes the pressure information measured by the sensor units 200 and the wireless communication module 340 wirelessly transmits balance correction information on the user to a data output unit 350 in a PC or the like or a display unit 350 in a smartphone or the like.

The wireless communication module 340 is a Wi-Fi communication module, a Bluetooth communication, or a Zigbee communication module.

Particularly, the use of a Bluetooth low energy (BLE) module as the Bluetooth communication module remarkably reduces power consumption

As described above, the balance measurement module 320 of the controller 300 computes the center of gravity on the basis of the pressure information measured by the sensor units 200 installed within the first, second, third, and fourth combination through-holes 110a, 110b, 110c, and 110d, respectively, in the outsole 100. Alternatively, the balance measurement module 320 performs balance evaluation, that is, evaluation of a change in the center of gravity using the pressure information on the user, which is measured by the sensor units 200 in the user's standing posture on the basis of the center of gravity computed from the pressure information on the selected portions. The controller 300 may be built into the outsole 100. In this case, the wireless communication module 340 is provided for wireless communication between the controller 300 and the display unit 350. Alternatively, the controller 300 may communicate with the display unit 350 in a wired manner.

The balance measurement module 320 of the controller 300 provides the user in real time with feedback regarding pressure information, correction information, and balance information on the portions of the user's sole by transmitting a signal for vibration, a signal for push alarm, or a signal for an image to the display unit 350 to which the controller 300 establishes a BLE connection.

The balance information according to the present disclosure is fed back for correction, as a result of measuring and evaluating the pressure distributions over both soles of the user when the user takes the standing posture. The sensor units 200 each have a preset measurement value ratio and a range of numerical values of measurements. The sensor units 200 determines whether or not the user takes a posture that falls within a preset pressure distribution or whether or not the weight of the user falls within a preset distribution when putting on the lifting shoes. Then, a result of the determination is provided in the form of vibration or audio signal to the display unit 350 or the output module 360 included in the controller 300 within the outsole 100. Alternatively, the result of the determination is displayed on the display unit 350.

In addition, a button that operates in conjunction with the controller 300 may be provided. When the user sits in a proper balance posture, a value of the proper balance posture is stored by pushing down the button.

The balance measurement module 320 of the controller 300 has functions of calculating the center of gravity, evaluating the ability to maintain a balance, storing data, and executing a training program. Using the sensor units 200, the balance measurement module 320 evaluates the ability of the user to maintain the balance. Guidance on training for barbel lifting is provided according to a result of the evaluation.

The controller 300 performs evaluation of the fundamental ability, that is, evaluation of the ability of the user to maintain the balance prior to training, evaluation of the ability to adapt to training, that is, evaluation of the ability of the user to maintain the balance during training, and evaluation of the ability to make an improvement, that is, evaluation of the ability of the user to maintain the improved balance after training. Thus, the evaluation and the analysis can be more systematically made of performance in training, that is, an improvement in the ability to maintain the balance.

According to an embodiment, the center of gravity is calculated by the pressure input module 310 or the balance measurement module 320 of the controller 300. A position of the center of gravity is calculated using values of a pressure (weight) and position of the user, which are measured by the sensor to units 300 installed within the combination through-holes 110a, 110b, 110c, and 110d, respectively, in the outsole 100. For example, it is assumed that the weight of the user is 100 kg when the user takes the standing posture. Accordingly, the sensor units are mounted within four combination through-holes, that is, the combinations holes 110a, 110b, 110c, and 110d, respectively, in the outsole 100 of the one lifting shoe that is put on. In this case, as reference information, it is determined that the sensor unit 200 in the first combination through-hole 110a is assigned 25 kg, the sensor unit 200 in the second combination through-hole 110b is assigned 15 kg, the sensor unit 200 in the third combination through-hole 110c is assigned 10 kg, and the sensor unit 200 in the fourth combination through-hole 110d is assigned 0 kg. A reference center of gravity is determined on the basis of the reference information. In this case, when the user who puts on the lifting shoes takes the standing posture, the position and weight of the user, which are measured by the four sensor units 200 are computed. Then, the weight and the center of gravity of the user are calculated using the reference information.

In addition to the embodiment directed to the reference center of gravity, the calculated center of gravity of the user may be used as data for the reference center of gravity. When the user who puts on the lifting shoes lifts a barbel while taking the standing posture that is determined considering his/her posture, lengths of his/her feet, shapes of his/her sole, and the like, the sensor units 200 within the combination through-holes 110a, 110b, 110c, and 110d, respectively, measure pressure values at the time and position at which he/she maintains an optimal balance state. Data on the center of gravity of the user, which is calculated on the basis of the measured pressure values, may be utilized as the reference center of gravity.

In order to evaluate the ability to maintain the balance, the controller 300 compares a positional value of the center of gravity of the user, which is calculated from the center of gravity of the user, with a positional value of the reference center of gravity, and calculates the pressure values measured by the sensor units 200, and thus evaluates a balance state of the user. Then, the pressure values recorded in the sensor units 200 are corrected until the measured positional value of the center of gravity of the user is the same as the positional value of the reference center of gravity. To do this, the adjustment enabling portions 400, each having an adjusted length, are closely connected into the combination through-holes 110a, 110b, 110c, and 110d, respectively, associated with the correction. Numerical values, such as pressure values, that are obtained through the sensor units 200 after the heights are adjusted are additionally reflected in evaluating the balance state of the user. In this manner, results of evaluating the ability of the user to maintain the balance are obtained. In addition, the results of the evaluation may be quantified and tabulated in the form of charts.

As described above, a proper balance state of the user is determined by comparing the center of gravity of the user with the reference center of gravity. However, the pressure value of each of the sensor units 200 installed within the outsole 100 of each of the lifting shoes put on by the user may be compared with a reference pressure value recorded in terms of medical science or kinematics. Alternatively, the pressure that is measured by each of the sensor units 200 when the user maintains the best record may be utilized as the reference pressure value. In this case, the heights of the adjustment enabling portions 400 closely connected into the combination through-holes 110a, 110b, 110c, and 110d within which the sensor units 200 associated with occurrence of a pressure difference are installed are adjusted, or the adjustment enabling portions 400 are replaced with adjustment enabling portions 400 having different lengths. In this manner, the balance state of the user is maintained.

An evaluation chart can be created by accumulating data on results of executing the training program that will be described below. In addition, a comprehensive testing result chart that shows the ability of the user to maintain the balance can be created on the basis of the accumulated data. That is, in order to evaluate the ability to maintain the balance, the controller 300 according to the present disclosure receives the degree to which the body of the user is imbalanced and the position of the center of gravity of the user, which are measured by the sensor units 200. Furthermore, the controller 300 performs comprehensive computation for conversion into an inclination angle of the body of the user and a value of the change in the center of gravity. In this manner, the ability of the user to maintain the balance and the balance state of the user are measured, evaluated, and calculated.

In a case where the user takes exercise according to the training program with varying levels of difficulty that is created on the basis of the evaluation chart, another evaluation chart may be created by tabulating results of barbell lifting training for the user, that is, results of evaluation of the ability of the user to adapt to training. Another evaluation chart may be created by comprehensively tabulating results of evaluation of the fundamental ability of the user and results of evaluation of the ability of the user to adapt to training after the barbell lifting training. Thus, results of evaluating the ability of the user that is improved after the barbell lifting training can be obtained.

Therefore, the user who puts on the lifting shoes can maintain a proper balance by taking the standing posture or by taking exercise in the standing posture after adjusting the height of the outsole 100 of each of the lifting shoes from the ground. In a case where, according to the pressure value measured by each of the sensor units 200, the user requests for the change in the center of gravity, or the change in the center of gravity is determined, the adjustment enabling portions 400 in the combination through-holes 110a, 110b, 110c, and 110d associated with the correction are replaced with new adjustment enabling portions 400 having different lengths. Thereafter, the ability of the user to maintain the balance is evaluated by the user or through the computation of the center of gravity by the controller 300. In this manner, the user can maintain a proper balance posture for taking exercise.

In addition, in order to adjust the heights of the first, second, third, and fourth combination through-holes 110a, 110b, 110c, and 110d in the outsole 100 from the ground with which the outsole 100 is brought into contact, the adjustment enabling portions 400 in the first, second, third, fourth combination through-holes 110a, 110b, 110c, and 110d are replaced with new adjustment enabling portions having different lengths. Pressure information measured by the sensor units 200 through adjustment enabling portions 400 closely connected into the first, second, the third, fourth combination through-holes 110a, 110b, 110c, and 110d, respectively, that are supported on the ground is compared by the controller 200 with preset reference pressure information. When it is determined that the correction has to take place, the adjustment enabling portions 400 can be replaced with new adjustment enabling portions 400 that have different lengths. In this manner, the user can take a proper posture for taking exercise, thereby maintaining his or her balance.

The new adjustment enabling portions 400 having different lengths may be closely connected into the first, second, third, and fourth combination through-holes 110a, 110b, 110c, and 110d in the outsole 100 on the basis of the balance information provided by the controller 300. Thus, with the pressure information measured in real time by the sensor units 200, the user can take a stabilized posture for taking exercise.

After the user puts on the lifting shoes, the sensor units 200 and terminal portions 500 of the adjustment enabling portions 400 in the combination through-holes 110a, 110b, 110c, and 110d are brought into close contact with each other such that the weight of the user is supported in a distributive manner on the ground. To this end, an adjustment enabling portion 400 is provided as follows.

First, as illustrated in FIGS. 6A and 6B, the adjustment enabling portion 400 is made of the same elastic and restorable material as the outsole 100 of the lifting shoe, or is made of the same material as the outsole 100 of the lifting shoe. In this case, the adjustment enabling portion 400 is transformed due to the weight of the user and is pressed against the ground. Thus, the terminal portion 500 is combined with a contact terminal (not illustrated) of the sensor unit 200 and the center of an upper part of the adjustment enabling portion 400 brought into the contact terminal of the sensor unit 200. The terminal portion 500 has a contact tab 510 protruding upward from the center, and a protrusion jaw 520 protruding downward from a circumferential end. The adjustment enabling portion 400 has a cylindrical insertion groove 430 in the upper part. The protrusion jaw 520 of the terminal portion 500 is inserted into the cylindrical insertion groove 430 for combination. The adjustment enabling portion 400 has a cylindrical edge portion 410, a cylindrical space groove 420, and a reinforcement tab 440 on an external circumferential end portion. The cylindrical space groove 420 is inwardly provided next to the cylindrical edge portion 410. The reinforcement tab 440 is positioned between the cylindrical space groove 420 and the cylindrical insertion groove 430.

The terminal portion 500 integrally includes the contact tab 510 protruding upward from the center and the protrusion jaw 520 in the shape of a cylindrical plate that is present in the vicinity of the contact tab 510. The cylindrical protrusion jaw 520 protrudes downward from the circumferential end.

The contact tab 510 of the terminal portion 500 protrudes from the center of an upper surface of the adjustment enabling portion 400 or is positioned on the same plane as an upper surface of the adjustment enabling portion 400.

The adjustment enabling portion 400 has the insertion groove 430 in the upper part. The protrusion jaw 520 of the terminal portion 500 is inserted into the cylindrical insertion groove 430 for combination. The adjustment enabling portion 400 has the cylindrical edge portion 410, the cylindrical space groove 420, and the reinforcement tab 440 on the external circumferential end portion of the upper end. The cylindrical space groove 420 is inwardly positioned next to the cylindrical edge portion 410. The reinforcement tab 440 is positioned between the cylindrical space groove 420 and the cylindrical insertion groove 430.

Accordingly, the adjustment enabling portion 400 contracts due to a pressure applied against the ground. Thus, the contact tab 510 of the termination 500 positioned on the center of the upper part of the adjustment enabling portion 400 on which the pressure is concentratedly applied is brought into close contact with and pushes the sensor unit 200. At this time, the cylindrical space groove 420 contracts, thereby applying a pressure in a radial direction. Thus, the contact tab 510 of the terminal portion 500 maintains a home position thereof. At the same time, the contraction of the cylindrical space groove 420 causes the circular edge portion 410 to apply a pressure upward. Thus, the contact tab 510 is brought into close contact with the sensor unit 200 without deviating from a home position thereof.

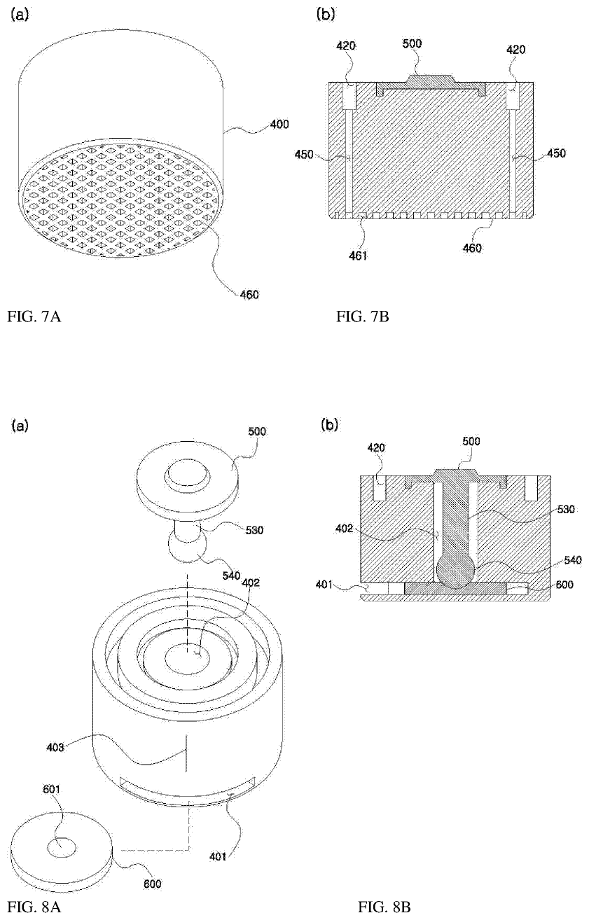

As illustrated in FIGS. 7A and 7B, a plurality of air flow through-holes 450 passes through the adjustment enabling portion 400 downward from a bottom of the cylindrical space groove 420 in such a manner to be positioned a distance away from each other.

The adjustment enabling portion has a net portion 460 on a lower surface. The net portions 460 have a plurality of grid grooves 461 arranged in matrix form. The plurality of grid grooves 461 communicate with the air flow through-holes 450. A cylindrical end portion of the adjustment enabling portion 400, which is positioned outward from the air flow through-holes 450 in the lower surface of the adjustment enabling portion 400, is bonded to an external circumferential end portion of the net portion 460 for connection. An internal surface of the adjustment enabling portion 400, which is positioned inward from the air flow through-holes 450, and an internal surface of an upper portion of the net portion 460 are brought into contact with each other without being bonded with each other.

The plurality of air flow through-holes 450 passes vertically through the adjustment enabling portion 400 from the bottom of the cylindrical space groove 420 in the upper part of the adjustment enabling portion 400 to a lower portion of the adjustment enabling portion 400 in such a manner as to be positioned an equal distance away from each other. A pressure is applied between the sensor unit 200 and the upper part of the adjustment enabling portion 400 brought into close contact with the sensor unit 200. An air pressure generated by the application of the pressure keeps the sensor unit 200 and the terminal portion 500 from being brought into contact with each other. In order to prevent this phenomenon, the generated air pressure is released through the air flow through-holes 450.

The presence of the net portion 460 on the lower surface of the adjustment enabling portion 400 reduces the effect of solid contact with the ground, and thus the weight of the user is transferred, as is, through the adjustment enabling portion 400. An internal surface of an upper portion of the net portion 460 is not bonded. The internal surface thereof is positioned inward from the air flow through-holes in a lower part of the adjustment enabling potion 400. That is, the internal surface of the upper portion of the net portion 460 and an internal surface of the lower part of the adjustment enabling portion 400, which faces the internal surface of the upper portion of the net portion 460, are brought into contact with each other without being bonded with each other. An external surface of the upper part of the net portion 460 is bonded with a corresponding external surface of the lower part of the adjustment enabling portion 400 for combination.

As illustrated in FIGS. 8A and 8B, the adjustment enabling portion 400 has an arc-shaped first insertion hole 401 extending horizontally into an external circumferential surface of the lower part. The first insertion hole 401 is open at a first surface and is closed at a second surface facing the first surface. The adjustment enabling portion 400 has a vertical second insertion hole 402 that communicates with the center of the upper part of the adjustment enabling portion 400 and a center of the first insertion hole 401. A vertical support bar 530 that is inserted into the second insertion hole 402 in the adjustment enabling portion 400 is connected to a center of a lower surface of the terminal portion 500. A sphere-shaped rotation guidance ball 540 is connected to a lower end of the support bar 530. A load support plate 600 is inserted into the first insertion hole 401 in the adjustment enabling portion 401. The load support plate 600 has a semi-sphere-shaped support groove 601 in the center. The rotation guidance ball 540 of the terminal portion 500 is accommodated within the semi-sphere-shaped support groove 601.

The adjustment enabling portion 400 has the first insertion hole 401 and the vertical insertion hole 402 in an external circumferential surface of the lower part. The first insertion hole 401 extends horizontally into the external circumferential surface of the lower part. The first insertion hole 401 is open at the first surface in the shape of a cut semi-arc and is closed at the second surface facing the first surface. The first insertion hole 401 has a cylindrical groove inside. The vertical second insertion hole 402 communicates with the center of the upper part of the adjustment enabling portion 400 and the center of the first insertion hole 401.

The vertical support bar 530 is integrally connected vertically to the center of the lower part of the terminal portion 500 and the sphere-shaped rotation guidance ball 540 is integrally connected to the lower end of the vertical support bar 530. The vertical support bar 530 is inserted into the second insertion hole 402 in the adjustment enabling portion 400.

The load support plate 600 has the semi-sphere-shaped support groove 601 in the center of an upper portion. The load support plate 600 is inserted in the first insertion hole 401 in the adjustment enabling portion 400. The rotation guidance ball 540 of the terminal portion 500 is accommodated within the semi-sphere-shaped support groove 601. With this structure, the rotation guidance ball 540 is supportable and rotatable in a state of being accommodated within the support groove 601.

The load support plate 6 maintains a state of being in parallel with the ground. A slope of the adjustment enabling portion 400 changes according to a change in a height of the sole of the user from the ground. At this time, the support bar 530 of the terminal portion 500 serves as a guide for concentratedly transferring the weight of the user through the adjustment enabling portion 500. At the same time, the support bar 530 remains positioned at the center of the load support plate 600. Thus, the weight of the user is concentratedly transferred, as is, to the terminal portion 500, thereby bringing the terminal portion 500 into close contact with the sensor unit 200.

As illustrated in FIGS. 8A and 8B, the adjustment enabling portion 400 has a plurality of cut lines 403, each having a predetermined vertical length, in an external circumferential surface of the center. The cut lines 403 are positioned an equal distance away from each other. The cut lines 403 possibly communicate with the second insertion hole 402. An air pressure that is generated by the sensor unit 200 and the upper part of the adjustment enabling portion 400 being brought into close contact with each other is released through the second insertion hole 402 to the cut lines 403. Thus, the adjustment enabling portion 400 has a shape corresponding to a slope with respect to the support bar 530. The cut lines 403, each having the vertical length, are arranged an equal distance away from each other in the center of an external circumferential surface of the adjustment enabling portion 400. When a pressure is not applied to the adjustment enabling portion 400, the cut lines that result from internally cutting the adjustment enabling portion 400 and possibly communicate with the second insertion hole 402 are brought into contact with each other. In contrast, when the pressure is applied to the adjustment enabling portion 400, the cut lines are separated a very small distance away from each other and thus communicate with the second insertion hole 402.

The heights of the adjustment enabling portions 400 in the combination through-holes 110a, 110b, 110c, and 110d selected for locating the center of gravity of the user, that is, maintaining his or her balance after the lifting shoes are put on are adjusted for correction. In this state, in order to stably maintain the balance of the user and to prevent a position of the adjustment enabling portion 400 from being changed, a stability maintenance portion 700 is further provided. The stability maintenance portion 700 is combined with a protruding part of the adjustment enabling portion 400. The stability maintenance portion 700 is combined with the protruding part of the adjustment enabling portion 400. The part thereof protrudes from each of the combination through-holes 110a, 110b, 110c, and 110d in the outsole 100.

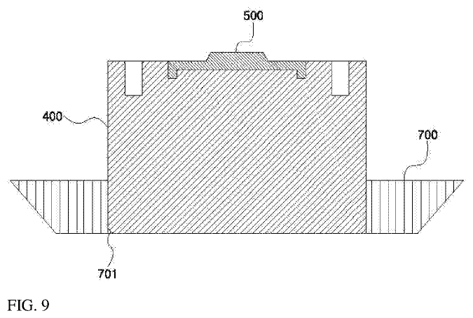

As illustrated in FIG. 9, the stability maintenance portion 700 is combined with the protruding part of the adjustment enabling portion 400 that is selected from among the adjustment enabling portions 400 closely connected into the first, second, third, and fourth combination through-holes 110a, 110b, 110c, and 110d, respectively. The part thereof protrudes from a bottom surface of the outsole 100. The stability maintenance portion 700 has the same height as the protruding part of the adjustment enabling portion 400. The stability maintenance portion 700 tapers from a first surface thereof that is brought into contact with the bottom surface of the outsole 100 to a second surface on which a protruding free end of the adjustment enabling portion 400 is positioned. The stability maintenance portion 700 has a fastening through-hole 701 that has the same height as the protruding part of the adjustment enabling portion 400 such that the protruding part thereof is fully inserted into the fastening through-hole 701.

Instead, the adjustment enabling portion 400 having an adjusted length is closely connected into the combination through-hole selected in association with the correction in order to maintain the balance state of the user. In this case, a protruding part of an adjustment enabling portion other than the adjustment enabling portions 400 closely connected into the combination through-holes 110a, 110b, 110c, and 110d, respectively, protrudes from a bottom surface of the outsole 100 and is combined with the stability maintenance portion 700.

The stability maintenance portion 700 prevents the protruding part of the adjustment enabling portion 400 from being deformed due to the weight of the user. That is, the stability maintenance portion 700 keeps the weight of the user distributed and thus keeps the shape of the protruding part maintained.

The protruding part of the adjustment enabling portion 400 is inserted closely into the fastening through-hole 701 in the stability maintenance portion 700. The protruding part of the adjustment enabling portion 400 has a cylindrical stepped hooking groove (not illustrated) in an external circumferential surface. The fastening through-hole 701 in the stability maintenance portion 700 has a protruding cylindrical hooking jaw (not illustrated) in an internal circumferential surface. A position of the protruding cylindrical hooking jaw in the internal circumferential surface corresponds to the cylindrical stepped hooking groove. Combination of the stability maintenance portion 700 and the adjustment enabling portion 400 engages the protruding cylindrical hooking jaw on the stability maintenance portion 700 with the cylindrical stepped hooking groove in the adjustment enabling portion 400. Thus, the stability maintenance portion 700 is prevented from deviating from the adjustment enabling portion 400. In addition, the stability maintenance portion 700 is integrally combined with the protruding part of the adjustment enabling portion 400.

Although the specific embodiment of the present disclosure has been described for illustrative purposes, those skilled in the art will appreciate that various modifications, additions and substitutions are possible, without departing from the scope and spirit of the disclosure as disclosed in the accompanying claims.

What is claimed is:

1. A smart lifting shoes for taking exercise, each of which is configured from an outsole to be brought into contact with the ground, a midsole combined with an upper portion of the outsole, an insole combined with an upper portion of the midsole to be brought into contact with a sole of a user, and an upper combined with the outsole to accommodate a foot of the user, the smart lifting shoes each comprising:
an outsole (100) configured to have first, second, third, and fourth combination through-holes (110a), (110b), (110c), and (110d) in positions thereon that correspond to a distal phalanx portion (P1), a first metatarsal bone portion (P2), a fifth metatarsal bone portion (P3), and a calcaneus portion (P4), respectively, of the sole, to have an accommodation groove (101) in a center of an upper part thereof, the center thereof corresponding to a center of the sole, and to have a connection groove (102) that connects between the accommodation groove (101) and the fourth combination through-hole (110d);
sensor units (200) installed within upper portions of the first, second, third, and fourth combination through-holes (110a), (110b), (110c), and (110d), respectively;
a controller (300) comprising:
a pressure reception module (310) mounted within the accommodation groove (101) for operation in conjunction with the sensor units (200) and receiving pressure information measured by the sensor units (200);
a balance measurement module (320) computing a balance of the sole on the basis of the pressure information and generating balance information;
a power supply module (330) supplying power;
a communication module (340) transmitting the pressure information and the balance information; and
adjustment enabling portions (400), each having a terminal portion (500) on an upper part thereof, that are closely connected into the first, second, third, and fourth combination through-holes (110a), (110b), (110c), and (110d), respectively, in the outsole (100), the terminal portion (500) serving for contact with each of the sensor units (200).

2. The smart lifting shoes of claim 1, wherein the sensor units (200) are installed within upper portions of the first, second, third, and fourth combination through-holes (110a), (110b), (110c), and (110d), respectively, in the outsole (100), and wherein each of the sensor units (200) is one of a force sensing resistor (FSR) or a load cell that measures a pressure, a weight, and a position.

3. The smart lifting shoes of claim 1, wherein the adjustment enabling portions (400), each with the terminal portion (500) for contact with each of the sensor units (200), are closely connected into the first, second, third, and fourth combination through-holes (110a), (110b), (110c), and (110d), respectively, in the outsole (100) , and wherein the terminal portions (500) are brought into contact with the sensor units (200), respectively, and thus the sensor units (200) measure pressure exerted on the adjustment enabling portions (400), respectively.

4. The smart lifting shoes of claim 1, wherein when a user putting on the smart lifting shoes takes a standing posture, the sensor units (200) measure pressure applied to the adjustment enabling portions (400) closely connected into the first, second, third, and fourth combination through-holes (110a), (110b), (110c), and (110d), respectively, and transmit the pressure information measured by the sensor units (200) that detect the pressure, to the controller (300) operating in conjunction with the sensor units (200),
wherein the controller (300) measures a pressure distribution over the sole of the user on the basis of the transmitted pressure information and analyzes the pressure,
wherein the controller further includes an output module (360) that operates in conjunction with the controller (300) under the control of the controller (300), and
wherein the output module (360) provides information for feedback in the form of vibration or audio according to information on the pressure distribution over the sole of the user, the information on the pressure distribution resulting from analyzing the pressure.

5. The smart lifting shoes of claim 1, wherein the balance measurement module (320) of the controller (300) performs functions of computing a center of gravity, evaluating ability to maintain a balance, storing data, and executing a training program.

6. The smart lifting shoes of claim 1, wherein the adjustment enabling portions (400) closely connected into the first, second, third, and fourth combination through-holes (110a), (110b), (110c), and (110d) are replaced with adjustment enabling portions (400) having different lengths, respectively, in order to adjust heights of upper surfaces of the first, second, third, and fourth combination through-holes (110a), (110b), (110c), and (110d) in the outsole (100) brought into contact with the ground, from the ground with which the outsole (100) is brought into contact.

7. The smart lifting shoes of claim 1, wherein the terminal portion (500) combined with a center of an upper part of one of the adjustment enabling portions (400) brought into contact with the sensor unit (200) has a contact tab (510) protruding from a center of an upper portion thereof, and a protrusion jaw (520) protruding from a lower cylindrical end thereof, and
wherein each of the adjustment enabling portions (400) has a cylindrical insertion groove (430) in the upper part and has a cylindrical edge portion (410), a cylindrical space groove (420), and a reinforcement tab (440) in an external circumferential end portion, the protrusion jaw (520) of the terminal portion 500 being inserted into the cylindrical insertion groove (430) for combination, the cylindrical space groove (420) being inwardly positioned next to the cylindrical edge portion (410), the reinforcement tab (440) being positioned between the cylindrical space groove (420) and the cylindrical insertion groove (430).

8. The smart lifting shoes of claim 7, wherein a plurality of air flow through-holes (450) passes through each of the adjustment enabling portions (400) downward from a bottom of the cylindrical space groove (420) in such a manner as be positioned a distance away from each other,
wherein each of the adjustment enabling portions (400) has a net portion (460) on a lower surface, the net portions (460) having a plurality of grid grooves (461) arranged in matrix form and communicating with the air flow through-holes (450),
wherein a cylindrical end portion of each of the adjustment enabling portions (400), which is positioned outward from the air flow through-holes (450) in a lower surface of the adjustment enabling portion (400) is bonded to an external circumferential end portion of the net portion (460) for connection, and
wherein an internal surface of each of the adjustment enabling portions (400), which is positioned inward from the air flow through-holes (450), and a corresponding internal surface of an upper portion of the net portion (460) are brought into contact with each other without being bonded with each other.

9. The smart lifting shoes of claim 1, wherein each of the adjustment enabling portions (400) has an arc-shaped first insertion hole (401) extending horizontally into an external circumferential surface of a lower part, the first insertion hole (401) being open at a first surface and being closed at a second surface facing the first surface,
wherein each of the adjustment enabling portions (400) has a vertical second insertion hole (402) in a center of an upper part, the vertical second insertion hole (402) communicating with a center of the first insertion hole (401) from top down,
wherein a vertical support bar (530) is connected to a center of a lower surface of the terminal portion (500) and is inserted into the second insertion hole (402) in the adjustment enabling portion (400),
wherein a sphere-shaped rotation guidance ball (540) is connected to a lower end of the vertical support bar (530), and
wherein a load support plate (600) inserted into the first insertion hole (401) in each of the adjustment enabling portions (400) has a semi-sphere-shaped support groove (601) in a center thereof, the rotation guidance ball (540) of the terminal portion 500 being accommodated within the semi-sphere-shaped support groove (601).

10. The smart lifting shoes of claim 9, wherein each of the adjustment enabling portion (400) has a plurality of cut lines (403), each having a predetermined vertical length, in an external circumferential surface of the center, the cut lines (403) being positioned an equal distance away from each other, and wherein the cut lines (403) communicate with the second insertion hole (402).

11. The smart lifting shoes of claim 1, wherein a stability maintenance portion (700) is combined with a protruding part of an adjustment enabling portion (400) selected from among the adjustment enabling portions (400) closely connected into the first, second, third, and fourth combination through-holes (110a), (110b), (110c), and (110d), respectively, the part thereof protruding from a bottom surface of the outsole (100),
wherein the stability maintenance portion (700) has the same height as the protruding part of the adjustment enabling portion (400),
wherein the stability maintenance portion (700) is tapered from a first surface thereof that is brought into contact with the bottom surface of the outsole (100) to a second surface on which a protruding free end of the adjustment enabling portion (400) is positioned, and
wherein the stability maintenance portion (700) has a fastening through-hole (701) that has the same height as the protruding part of the adjustment enabling portion (400) such that the protruding part thereof is fully inserted into the fastening through-hole (701).
